(19) United States
(12) Patent Application Publication
(10) Pub. No.: US 2012/0028346 A1
(43) Pub. Date: Feb. 2, 2012

(54) Microalgae Photobioreactor

(76) Inventor: David A. Hazlebeck, El Cajon, CA (US)

(21) Appl. No.: 12/845,489

(22) Filed: Jul. 28, 2010

(51) Int. Cl. C12M 1/00; B32B 37/02

(52) U.S. Cl. 435/292.1; 435/289.1; 156/60

(57) ABSTRACT

A bioreactor is provided for circulating a fluid medium. To reduce manufacturing and maintenance expenses, the bioreactor is formed from two sheets of transparent plastic. Structurally, each sheet has first and second edges extending in an axial direction between proximal and distal ends. To form the bioreactor, the sheets are sealed to one another along their respective first edges, distal ends, and second edges. Also, the sheets are sealed to one another along an axially-extending boundary positioned between the first and second edges. As a result, a substantially U-shaped channel is defined between the first and second sheets. Further, two side-by-side openings to the channel are defined by the proximal ends of the sheets. Also, the bioreactor includes a conduit interconnecting the first opening and the second opening. A pump is positioned in the conduit to circulate the fluid medium through the channel.

### FIELD OF THE INVENTION

The present invention pertains generally to photobioreactors. More particularly, the present invention pertains to photobioreactors that circulate a fluid medium for growing microalgae. The present invention is particularly, but not exclusively, useful as a bioreactor that includes a fluid medium circulation channel formed by sealing two plastic sheets together at selected locations.

### BACKGROUND OF THE INVENTION

The growth rate of selected microalgae in a liquid environment is dependent on several disparate factors. For one, it is known that the fluid medium in which the selected microalgae grows (i.e. liquid environment) must be circulated to provide for mixing and exposure of the selected microalgae to light for photosynthesis. For another, the selected microalgae may require protection from competition with contaminants such as other algae, bacteria, and viruses. Also, loss of fluid medium through evaporation should be minimized or compensated for. Due to their success in confronting these issues, closed systems, such as photobioreactors, are frequently used to effectively grow microalgae.

However, closed photobioreactors are typically complicated devices that include multiple connections, high pressure drops, expensive materials, and vertical supports. As a result, typical closed photobioreactors are expensive to construct, maintain and operate.

In light of the above, it is an object of the present invention to provide a closed photobioreactor that minimizes required connections. Another object of the present invention is to provide a photobioreactor having a channel formed by selectively sealing two transparent plastic sheets together. Still another object of the present invention is to provide a photobioreactor for promoting microalgae growth that relies on gravity as the primary force for moving a fluid medium through the photobioreactor. Yet another object of the present invention is to provide a photobioreactor that is relatively simple to manufacture, is easy to use, and is comparatively cost effective.

### SUMMARY OF THE INVENTION

A bioreactor that is used to circulate a fluid medium for the purpose of growing algae includes a substantially U-shaped channel having a first and second opening. Further, the channel is interconnected with a conduit formed in a transfer section. Specifically, the transfer section includes a collection trough connected to the second opening and an elevated distribution trough connected to the first opening. Between the collection trough and the distribution trough, a pump or other flow device is positioned to drive the fluid medium from the collection trough to the distribution trough.

Importantly, the U-shaped channel is formed from two transparent plastic sheets. Structurally, each sheet has a proximal end, a distal end, and side edges extending between the ends. For purposes of the present invention, the sheets are bonded to one another at an outer seal along their respective side edges and distal ends. Further, the sheets are bonded to one another at an inner seal along a longitudinally-extending median located between the opposite side edges. Because the inner seal does not intersect the outer seal, the U-shaped channel is formed between the two sheets. Further, the first and second openings of the channel are positioned adjacent one another along the proximal end of the sheets.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of a bioreactor in accordance with the present invention;

FIG. 2 is a perspective view of the sheets used to form the channel of the bioreactor shown in FIG. 1;

FIG. 3A is an overhead view of the sheets of FIG. 2 during formation of the channel;

FIG. 3B is an overhead view of an alternate formation of the channel from the sheets; and

FIG. 4 is a perspective view of the bioreactor, illustrating the function of the transfer section in accordance with the present invention.

### DESCRIPTION OF THE PREFERRED EMBODIMENTS

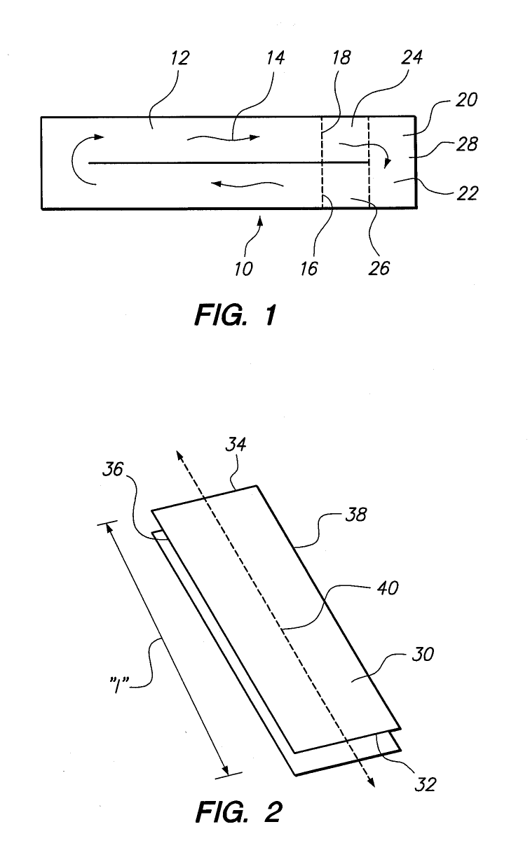

Referring initially to FIG. 1, a bioreactor is shown, and is generally designated 10. As shown in FIG. 1, the bioreactor 10 forms a U-shaped channel 12 which holds a fluid medium (arrow 14). For purposes of the present invention, the channel 12 has a first opening 16 and a second opening 18. As shown, a transfer section 20 forming a conduit 22 interconnects the first opening 16 and the second opening 18. Structurally, the transfer section 20 includes a collection trough 24 for receiving the fluid medium 14 from the second opening 18. Further, the transfer section 20 includes a distribution trough 26 for feeding the fluid medium 14 to the first opening 16. Interconnected between the collection trough 24 and the distribution trough 26 is a device 28 for flowing the fluid medium 14.

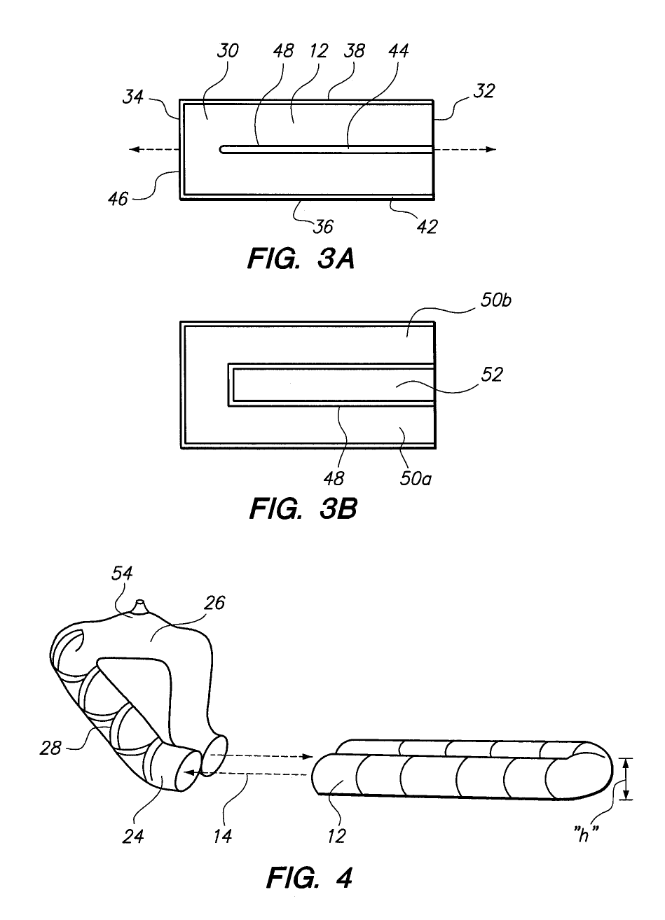

Referring now to FIG. 2, the construction of the U-shaped channel 12 may be understood. As shown, the channel 12 is formed from two transparent plastic sheets 30. Each sheet 30 has a proximal end 32, a distal end 34, a first edge 36 and a second edge 38. Further, the sheets 30 define a longitudinal axis 40 and have a length “I” that may be between 0.5 and 2.0 kilometers. Cross-referencing FIG. 2 with FIG. 3A, it can be seen that the sheets 30 are bonded to one another by a seal 42 along the first edges 36, the distal ends 34, and the second edges 38. Further, the sheets 30 are bonded together by an axially-extending seal 44 that is distanced between the first and second edges 36, 38. As a result, the seal 42 forms an outer boundary 46 and the seal 44 forms an inner boundary 48 for the U-shaped channel 12. In FIG. 3B, an alternate embodiment is shown in which the inner boundary 48 itself is U-shaped. As a result, the parallel legs 50a, 50b of the channel 12 are separated from one another by a void 52. As a result, increased sunlight may enter each leg 50a, 50b.

As illustrated in FIG. 4, the channel 12 is substantially circular, though it may be flat-bottomed, or rectangular. Further, it can be seen that the channel 12 has a height “h” which is less than 15 centimeters, and preferably about 7.5 centimeters. As shown in FIG. 4, the flow device 28 is an Archimedes pump that elevates the fluid medium 14 from the collection trough 24 to the distribution trough 26. Alternatively, the flow device 28 may be a conveyor, a bucket lift, a paddle wheel, a sealed paddle wheel, an electro-mechanical pump, or a similar device for moving fluid. As further shown in FIG. 4, the bioreactor 10 includes a disentrainment section 54 for removing gas such as oxygen from the fluid medium 14. The gas may be captured as a product or released.

While in FIGS. 1, 3A, 3B and 4, the bioreactor 10 is illustrated to include a single seal 44, it is noted that multiple seals 44 can be used to establish a longer channel 12. Despite the length of the channel 12, the present bioreactor 10 utilizes only two connections, from the distribution trough 26 to the first opening 16 and from the second opening 18 to the collection trough 24. Therefore, the costs associated with manufacturing and maintaining interconnections are reduced. Further, the present bioreactor 10 relies on a single flow device 28 which can supply acres of fluid medium flow.

For use of the bioreactor 10, the collection trough 24 is connected to the second opening 18 and the distribution trough 26 is connected to the first opening 16. Then, the flow device 28 is activated and pumps the fluid medium 14 from the collection trough 24 to the distribution trough 26. As gravity pulls the fluid medium 14 from the distribution trough 26 to the first opening 16, circulation of the fluid medium 14 through the channel 12 and conduit 22 is established.

While the particular Microalgae Photobioreactor as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A bioreactor for circulating a fluid medium which comprises:
a first sheet and a second sheet, with each sheet extending along an axis from a proximal end to a distal end and having a first edge and a second edge interconnecting the ends, wherein the first edges of the sheets are sealed together, the distal ends of the sheets are sealed together, and the second edges of the sheets are sealed together, wherein the sheets are further sealed together along an axially-extending inner boundary distanced between the first and second edges to define a substantially U-shaped channel having a first opening and a second opening at the proximal end of the sheets;
a transfer section connecting the second opening of the channel in fluid communication with the first opening of the channel; and
a lifting device for lifting fluid medium in the transfer section from the second opening of the channel and for providing gravity-driven fluid medium to the first opening of the channel to circulate the fluid medium through the bioreactor.

2. A bioreactor as recited in claim 1 wherein the transfer section comprises a collection trough connected to the second opening of the channel and a distribution trough connected to the first opening of the channel, and wherein the lifting device is interconnected between the collection trough and the distribution trough.

3. A bioreactor as recited in claim 2 wherein the distribution trough is elevated above the channel.

4. A bioreactor as recited in claim 3 wherein the lifting device is selected from the group consisting of an Archimedes pump, a conveyor, a bucket lift, a paddle wheel, a sealed paddle wheel and an electro-mechanical pump.

5. A bioreactor as recited in claim 1 wherein the transfer section includes a disentrainment section for removing gas from the fluid medium.

6. A bioreactor as recited in claim 1 wherein the sheets are transparent, wherein the channel forms two substantially parallel legs, and wherein the inner boundary is substantially U-shaped to define a void between the legs for transmitting sunlight to the fluid medium in the legs.

7. A bioreactor for circulating a fluid medium which comprises:
a first sheet sealed to a second sheet along an outer boundary and along an inner boundary to define a substantially U-shaped channel having a first opening and a second opening; and
a conduit connecting the second opening of the channel in fluid communication with the first opening of the channel, wherein the conduit includes a means for flowing the fluid medium from the second opening to the first opening to circulate the fluid medium through the channel.

8. A bioreactor as recited in claim 7 wherein the conduit comprises a collection trough connected to the second opening of the channel and a distribution trough connected to the first opening of the channel, and wherein the flowing means is interconnected between the collection trough and the distribution trough.

9. A bioreactor as recited in claim 8 wherein the distribution trough is elevated above the channel, and wherein the flowing means is a device that lifts fluid medium from the collection trough to the distribution trough.

10. A bioreactor as recited in claim 9 wherein the lifting device is selected from the group consisting of an Archimedes pump, a conveyor, a bucket lift, a paddle wheel, a sealed paddle wheel and an electro-mechanical pump.

11. A bioreactor as recited in claim 7 wherein the conduit includes a disentrainment section for removing gas from the fluid medium.

12. A bioreactor as recited in claim 7 wherein the sheets are transparent, wherein the channel forms two substantially parallel legs, and wherein the inner boundary is substantially U-shaped to define a void between the legs for transmitting sunlight to the fluid medium in the legs.

13. A bioreactor as recited in claim 7 wherein the channel has a height “h” that is less than 15 centimeters.

14. A bioreactor as recited in claim 13 wherein the height is 7.5 centimeters.

15. A bioreactor as recited in claim 7 wherein the sheets have a length “I” that is between 0.5 kilometers and 2.0 kilometers.

16. A method for constructing a bioreactor for circulating a fluid medium comprising the steps of:
providing a first sheet and a second sheet, with each sheet extending along an axis from a proximal end to a distal end and having a first edge and a second edge interconnecting the ends;
sealing the sheets together along their respective first edges, distal ends and second edges;
bonding the sheets together along an axially-extending inner boundary distanced between the first and second edges to define a substantially U-shaped channel having a first opening and a second opening at the proximal end of the sheets;
interconnecting a transfer section between the second opening and the first opening; and
positioning a lifting device in the transfer section to lift fluid medium from the second opening of the channel and to provide gravity-driven fluid medium to the first opening of the channel to circulate the fluid medium through the bioreactor.

17. A method as recited in claim 16 wherein the transfer section comprises a collection trough connected to the second opening of the channel and a distribution trough connected to the first opening of the channel, and wherein the lifting device is interconnected between the collection trough and the distribution trough.

18. A method as recited in claim 17 further comprising the step of elevating the distribution trough above the channel.

19. A method as recited in claim 16 further comprising the step of establishing a disentrainment section in the transfer section for removing gas from the fluid medium.

20. A method as recited in claim 16, wherein the sheets are transparent, wherein the channel forms two substantially parallel legs, and wherein, during the bonding step, a void is defined between the legs for transmitting sunlight to the fluid medium in the legs.
